(19) United States
(12) Patent Application Publication
(10) Pub. No.: US 2009/0299120 A1
(43) Pub. Date: Dec. 3, 2009

(54) PROCESS FOR PRODUCING PURIFIED GAS

(76) Inventors: Henricus Abraham GEERS, Rijswijk (NL); William David Prince, Aberdeen (GB)

Correspondence Address: SHELL OIL COMPANY, P O BOX 2463, HOUSTON, TX, 772522463, US

(21) Appl. No.: 12/473,545

(22) Filed: May 28, 2009

(30) Foreign Application Priority Data
May 30, 2008 (EP) 08157277.8

(51) Int. Cl. C07C 7/10; C07C 7/14

(52) U.S. Cl. 585/815; 585/833

(57) ABSTRACT

Process for producing purified hydrocarbon gas from a gas stream comprising methane and acidic contaminants, which process comprises the steps of cooling the gas stream in a first cooling stage to a first temperature to form a first mixture of solid and/or liquid acidic contaminants and a vapour containing gaseous hydrocarbons and a reduced amount of acidic contaminants; separating the solid and/or liquid acidic contaminants from the first mixture, yielding partly purified gas; cooling the partly purified gas in a second cooling step to a second temperature to form a second mixture comprising purified hydrocarbon gas and further solid and/or liquid acidic contaminants; and separating the further solid and/or liquid acidic contaminants from the second mixture, yielding the purified hydrocarbon gas.

### RELATED APPLICATIONS

The present application claims priority to co-pending European Patent Application number 08157277.8-1213, filed on May 30, 2008, and having attorney docket number TS6930 EPC. European Patent Application number 08157277.8 is herein incorporated by reference in its entirety.

### FIELD OF THE INVENTION

The present invention relates to a process for producing purified gas. The invention especially relates to a process in which purified gas is produced from natural gas containing carbon dioxide and hydrogen sulphide and optionally other acidic contaminants.

### BACKGROUND OF THE INVENTION

Such a process is known from WO-A 2004/070297. This document discloses a process in which a natural gas stream comprising methane and acidic contaminants is first cooled to remove water from the natural gas, and subsequently the natural gas is further cooled to solidify acidic contaminants or dissolve such contaminants in a liquid, which contaminants are removed so that a purified natural gas is recovered.

It has been found that this process is very suitable when the natural gas stream contains relatively small amounts of acidic contaminants, such as up to 25 % vol. However, there is room for improvement of this process when the natural gas streams contain high concentrations, i.e. at least 25 volume %, of acidic contaminants.

A two step process is known from WO-A 2007/030888, which document discloses a process in which a natural gas stream comprising methane and acidic species is dehydrated and subsequently cooled to obtain a slurry of solid acidic contaminants and liquid hydrocarbons together with a gaseous stream containing gaseous acidic species. The slurry is removed and the gaseous stream containing the gaseous acidic species is treated with a solvent, e.g., methanol, to wash the gaseous acidic species from the gaseous stream, resulting in a purified natural gas product. The acidic species are contained in the solvent, and are recovered from the solvent in a subsequent desorption step. The solvent may be recycled to the wash treatment after a number of heat exchange steps.

This process requires a cumbersome recovery of the solvent in a desorption step and it also requires heat exchange steps before recycling the solvent to the wash treatment.

### SUMMARY OF THE INVENTION

It has now been found that an efficient removal of acidic contaminants from gases such as natural gas with a high content of acidic contaminants can be obtained without the need for a complex wash unit and expensive adsorption/desorption steps.

Accordingly, the invention provides a process for producing purified hydrocarbon gas from a gas stream comprising methane and acidic contaminants, which process comprises the steps of:
(a) cooling the gas stream in a first cooling stage to a first temperature to form a first mixture of solid and/or liquid acidic contaminants and a vapour containing gaseous hydrocarbons and a reduced amount of acidic contaminants;
(b) separating the solid and/or liquid acidic contaminants from the first mixture, yielding partly purified gas;
(c) cooling the partly purified gas in a second cooling step to a second temperature to form a second mixture comprising purified hydrocarbon gas and further solid and/or liquid acidic contaminants; and
(d) separating the further solid and/or liquid acidic contaminants from the second mixture, yielding the purified hydrocarbon gas.

### BRIEF DESCRIPTION OF THE FIGURE

FIG. 1 shows a schematic flow scheme of an embodiment according to the invention.

FIG. 2 shows a more detailed view of one of the vessels of FIG. 1.

### DETAILED DESCRIPTION OF THE INVENTION

The present process provides a solution to the purification of gas streams that contain relatively large amounts of acidic contaminants. In the first cooling stage a large proportion of the acidic contaminants are solidified and/or liquefied and the thus formed solids and/or liquids are subsequently removed, whereas the partly purified gas contains the gaseous hydrocarbons and a reduced amount of vaporous acidic contaminants. Because a substantial amount of acidic contaminants, representing a potentially significant portion of the gas stream, has been removed in the first cooling stage, a smaller amount of gas needs to be cooled in the second cooling stage in order to solidify and/or liquefy further acidic contaminants. Due to the fact that in the second cooling stage a smaller amount of gas is to be cooled down, the required energy is less than when the entire gas stream had to be cooled down. In this way a better removal of acidic contaminants is obtained and the losses of hydrocarbons are reduced. Furthermore, as the process is conducted in two or more stages it offers more flexibility.

The gas stream can be any stream of gas that comprises acidic contaminants and hydrocarbons. In particular the process according to the present invention can be applied to a natural gas stream, i.e., a gas stream that contains significant amounts of methane and that has been produced from a subsurface reservoir. It includes a methane natural gas stream, an associated gas stream or a coal bed methane stream. The amount of the hydrocarbon fraction in such a gas stream is suitably from 10 to 85 mol % of the gas stream, preferably from 25 to 75 mol %. Especially the hydrocarbon fraction of the natural gas stream comprises at least 75 mol % of methane, preferably 90 mol %. The hydrocarbon fraction in the natural gas stream may suitably contain from 0 to 20 mol %, suitably from 0.1 to 10 mol %, of C₂-C₆ compounds. The gas stream may also comprise up to 20 mol %, suitably from 0.1 to 10 mol % of nitrogen, based on the total gas stream.

In the process of the invention the acidic contaminants are in particular hydrogen sulphide and/or carbon dioxide. It is observed that also minor amounts of other contaminants may be present, e.g. carbon oxysulphide, mercaptans, alkyl sulphides and aromatic sulphur-containing compounds. The major part of these components will also be removed in the process of the present invention.

The amount of hydrogen sulphide in the gas stream containing methane is suitably in the range of from 5 to 40 volume % of the gas stream, preferably from 20 to 35 volume % and/or the amount of carbon dioxide is in the range of from 10 to 90 vol %, preferably from 20 to 75 vol %, based on the total gas stream. It is observed that the present process is especially suitable for gas streams comprising large amounts of contaminants, e.g. 10 vol % or more, suitably between 15 and 90 vol %.

Gas stream containing the large amounts of contaminants as described above cannot be processed using conventional techniques as amine extraction techniques as they will become extremely expensive, especially due to the large amounts of heat needed for the regeneration of loaded amine solvent.

As indicated above, acidic contaminants that are usually present in natural gas streams include hydrogen sulphide and carbon dioxide. It is also possible that a natural gas stream contains other components, including ethane, propane and hydrocarbons with four or more carbon atoms. It will be appreciated that when a portion of acidic contaminants, e.g., carbon dioxide, solidifies and/or liquefies in the cooling stages, other components, e.g., hydrogen sulphide and hydrocarbons, may liquefy. The liquid components are suitably removed together with the solid and/or liquid acidic contaminants from the vapour.

It is immediately evident to the skilled person that the present process is different from the process described in WO-A 2004/070297. In the latter process a first cooling stage is carried out to create gas hydrates. To accomplish this it is explicitly observed that the temperature must be above the temperature at which solids of acidic species, such as hydrogen sulphide and carbon dioxide, are formed. In the present process the cooling is such that these acidic contaminants are liquefied or solidified. Preferably, the first and second temperature as defined above are at most the freeze out temperature of the first acidic contaminant to freeze out, the freeze out temperature being the temperature at which solid contaminants are formed. The skilled person will realize that the freeze out temperature may vary depending on the prevailing pressure.

The gas stream, and in particular natural gas streams produced from a subsurface formation, may typically contain water. In order to prevent the formation of gas hydrates in the present process, at least part of the water is suitably removed. Therefore, the gas stream that is used in the present process has preferably been dehydrated. This can be done by conventional processes. A suitable process is the one described in WO-A 2004/070297. Other processes for forming methane hydrates or drying natural gas are also possible. Other dehydration processes are also possible, including treatment with molecular sieves or drying processes with glycol or methanol. Suitably, water is removed until the amount of water in the gas stream comprises at most 50 ppmw, preferably at most 20 ppmw, more preferably at most 1 ppmw of water, based on the total gas stream.

In order to optimizes the solidification and/or liquefaction of further acidic contaminants in the second cooling stage, the second temperature in the second cooling stage is preferably lower than the first temperature in the first cooling stage. The skilled person can easily determine what the optimal temperature difference can be. Factors that influence the desired temperature difference include the amount of acidic contaminants in the gas stream and in the partly purified gas, the desired level of contaminants in the purified hydrocarbon gas, the nature of the acidic contaminants and other process conditions, including the pressures. Suitably the temperature difference between the first temperature and the second temperature amounts to 5 to 50° C. In order to liquefy and/or solidify a suitable amount of acidic contaminants, the first temperature is advantageously from −40 to −80 C, and the second temperature is from −50 to −100° C. These preferred temperatures provide suitable conditions for acidic contaminants to at least partly solidify.

In a first step of the present process the gas stream is cooled. The cooling may be effected by any known method, such as indirect heat exchange and expansion. Alternatively, a direct heat exchange, e.g., by spraying with a cold liquid, as shown in WO-A 2004/070297, is also possible. The skilled person will appreciate that expansion causes a lowering of temperature, so that cooling may be achieved by expansion and adapting pressure. In the present process it is preferred to use the energy for cooling as efficiently as possible. Therefore, the first cooling stage preferably comprises one or more heat exchange and/or expansion steps. Preferably the expansion is done by isenthalpic expansion, preferably isenthalpic expansion over an orifice or a valve, especially a Joule-Thomson valve or a series of Joule-Thomson valves. In another preferred embodiment the expansion is done by nearly isentropic expansion, especially by means of an expander, preferably a turbo expander, or a laval nozzle. Preferably, the gas stream is subjected to heat exchange with one or more cold process streams or external streams. Cold external streams may be suitable streams from an LNG (liquefied natural gas) plant, such a cold LNG stream or a refrigerant stream, or from an air separation unit. A suitable heat exchange step is between the gas stream and the partly purified gas exiting the first cooling stage. Another suitable heat exchange can be effected between the gas stream and the solid and/or liquid acidic contaminants that are separated from the aforesaid vapour.

Gas streams, such as natural gas streams, may become available at a temperature of −5 to 150° C. and a pressure of 10 to 700 bar, suitably from 20 to 200 bar. Although indirect heat exchange may be effective to accomplish significant cooling of the gas stream, it is preferred that the first cooling stage comprises one or more expansion steps. These expansion steps may be done via a Joule-Thomson valve, a venturi tube or a turbo-expander or any other expansion means known in the art.

As indicated above, the cooling eventually leads to liquid and preferably solid acidic contaminants. It is preferred to achieve the cooling in several steps, e.g., by indirect heat exchange, direct heat exchange by spraying with a cold liquid and/or expansion. Suitably, solid and/or liquid acidic contaminants are obtained in a final expansion step. The final expansion step is preferably achieved over a Joule-Thomson valve. Therefore, preferably, in a first step, which may be achieved by various intermediate steps and various methods, the gas stream is cooled to a temperature ranging from 1 to 40° C. above the freeze out temperature of the first acidic contaminant to freeze out, the freeze out temperature being the temperature at which solid contaminants are formed. Preferably, the cooling is effected till from 2 to 10° C. above the freeze out temperature. In a final step the gas stream is preferably cooled to the temperature at which a mixture of solid and/or liquid acidic contaminants and a vapour comprising gaseous hydrocarbons are formed by expansion over a valve. Preferably, the gas stream is partly or completely liquid before being expanded over the valve, and solid contaminants are formed upon expansion. This ensures a better separation performance in the vessel. Suitably, the gas stream is expanded from a pressure ranging from 20 to 200 bar to a pressure of 10 to 40 bar. Expansion over this pressure range suitably causes that liquid and/or solid acidic contaminants are formed.

The liquefaction and/or solidification of acidic contaminants may take place very rapidly, especially upon expansion over a valve, thereby forming the first mixture. To facilitate the separation the mixture is passed into a vessel, wherein the separation between solid and/or liquid acidic contaminants and vapour can take place. By gravity the solid acidic contaminants, and any other liquid that is formed, drops to the bottom of the vessel. After such separation the solid and/or liquid acidic contaminants are removed from the process. Since it is easier to transport liquids than to transport solids, it is preferred to melt at least partly the solid acidic contaminants, if formed. Such melting can be accomplished by heating the solids in the vessel by means of an electric immersion heater, by a bundle coil, i.e. a type of indirect heat exchanger, through which a process stream is fed, or by injecting a relatively warm fluid, such as a condensate. These measures have been suggested in WO-A 2004/0702897 and WO-A 2007/030888. In the present process it is preferred to heat at least a part of the withdrawn contaminants in a liquid, solid or slurry phase, and recycle at least a part of thus heated contaminants, in liquid or gaseous phase, to the vessel. In this way no extraneous material is recycled to the vessel. Preferably, all solid acidic contaminants are melted. In this way a liquid stream of contaminants is obtained, which can be easily transported further.

In a preferred embodiment, step (d) is performed in a separation vessel and is done using the steps of:
(d1) introducing a stream comprising liquid acidic contaminants into the intermediate or the bottom part or both of the separation vessel to obtain a diluted slurry of acidic contaminants;
(d2) introducing the diluted slurry of acidic contaminants via a slurry pump, preferably an eductor, into a heat exchanger in which solid acidic contaminant present in the diluted slurry of contaminants is melted into liquid acidic contaminant, wherein the heat exchanger is positioned outside the separation vessel, and the slurry pump, preferably the eductor, is arranged inside or outside the separation device or partly inside and outside the separation vessel;
(d3) introducing part or all of the liquid contaminant obtained in step d2 into a gas-liquid separator, wherein the gas-liquid separator is preferably the bottom part of the separation vessel;
(d4) introducing part or all of the liquid contaminant obtained in step d3 into the separation vessel as described above;
(d5) removing from the gas-liquid separator a stream of liquid acidic contaminant; and optionally
(d6) separating the stream of liquid contaminant obtained in step d5 into a liquid product stream and a recirculation stream which is used as a motive fluid in the eductor in the case that an eductor is used.

In this preferred embodiment, a continuously moving slurry phase is obtained, minimizing the risk of any blockages in the cryogenic separation vessel or in the pipelines and other pieces of equipment. Further, when a fully liquid stream is withdrawn from the heat exchanger, the absence of solid contaminant reduces the risk of blockages or erosion in subsequent pipelines or other equipment, and no damages will occur to any devices having moving parts, such as pumps. Moreover, when a pure liquid stream is withdrawn from the heat exchanger, a relatively cold liquid stream is obtained, thus minimizing the heat requirements of the separation device, and maintaining a high amount of exchangeable cold in the product stream.

In the event that the contaminant-rich stream mainly comprises carbon dioxide and is therefore a CO₂-rich stream, preferably CO₂-rich stream is further pressurized and injected into a subterranean formation, preferably for use in enhanced oil recovery or for storage into an aquifer reservoir or for storage into an empty oil reservoir. It is an advantage that a liquid CO₂-rich stream is obtained, as this liquid stream requires less compression equipment to be injected into a subterranean formation.

The partly purified gas that exits the first cooling and separation stage may be subjected to further cooling, or any other method to solidify or liquefy further acidic contaminants. When the cooling in the first stage has been done via indirect heat exchange only, such cooling for the second cooling stage may be effected by expansion. However, when already in the first cooling stage an expansion step has been applied, the partly purified gas becomes available at a reduced pressure for which it is not suitable to expand it further. It has been found that a better and more efficient separation of further acidic contaminants is obtainable if the partly purified gas is recompressed. Such recompressing can be done after heat exchange, e.g. with the gas stream as indicated above. Preferably, the partly purified gas is compressed in one or more compression steps. In order to make optimal use of the energy that is released at an earlier expansion step, the energy that is recovered at such expansion step or steps of the natural gas stream is preferably used for the compression step or steps of the partly purified gas. Since the volume of partly purified gas is smaller than that of the natural gas stream the expansion energy can compensate at least a significant part of the required compression energy.

The partly purified gas is preferably brought to a temperature ranging from 1 to 40 C, preferably 2 to 20 C above the freeze out temperature of the first acidic contaminant to freeze out, the freeze out temperature being the temperature at which solid contaminants are formed. As indicated above, the freeze out temperature also depends on the prevailing pressure. Hence, if the partly purified gas has been reheated, e.g., by heat exchange with the gas stream, cooling may be appropriate, e.g., by means of indirect heat exchange. The pressure may be adapted accordingly.

Although it is possible to cool by direct heat exchange, e.g., by spraying with a cold liquid, as shown in WO-A 2004/070297, or indirect heat exchange, it is preferred that the second cooling stage comprises one or more expansion stages. Like in the case of the first cooling stage the expansion can be achieved over a Joule-Thomson valve, a venturi tube, a turbo-expander or any other suitable expansion means that accomplishes a cooling of the partly purified gas. The use of a Joule-Thomson valve is preferred. Preferably, the partly purified gas is partly or completely liquid before being expanded over the valve, and solid contaminants are formed upon expansion. This ensures a better separation performance in the vessel. As indicated above, the second temperature obtained after the second cooling stage suitably amounts to −50 to −100 C. When the partly purified gas has been reheated due to compression and cooled by heat exchange and/or expansion, the partly purified gas is preferably expanded from a pressure ranging from 30 bar to 100 bar to a pressure of 5 to 30 bar.

The purified hydrocarbon gas that is being recovered after the final separation step can be used as product. It is also possible that it is desirable to subject the recovered purified hydrocarbon gas to further treatment and/or purification. The recovered purified hydrocarbon gas may also be subjected to further treatment and/or purification. For instance, the purified hydrocarbon gas may be subjected to fractionation. In the event that the purified hydrocarbon gas is natural gas intended for pipeline transportation or for producing liquefied natural gas (LNG), in order to reach pipeline specifications or LNG specifications the purified natural gas may further purified. Further purification can for example be done in an additional cryogenic distillation column, suitably with a bottom temperature between −30 and 10° C., preferably between −10 and 5° C. A reboiler may be present to supply heat to the column. Suitably the top temperature column is between −110 and −80° C., preferably between −100 and −90° C. In the top of the cryogenic distillation column a condenser may be present, to provide reflux and a liquefied (LNG) product.

As an alternative, further purification may be accomplished by absorption with a suitable absorption liquid. Suitable absorbing liquids may comprise chemical solvents or physical solvents or mixtures thereof.

A preferred absorbing liquid comprises a chemical solvent and/or a physical solvent, suitably as an aqueous solution.

Suitable chemical solvents are primary, secondary and/or tertiary amines, including sterically hindered amines.

A preferred chemical solvent comprises a secondary or tertiary amine, preferably an amine compound derived from ethanolamine, more especially DIPA, DEA, MMEA (monomethyl-ethanolamine), MDEA (methyldiethanolamine) TEA (triethanolamine), or DEMEA (diethyl-monoethanolamine), preferably DIPA or MDEA. It is believed that these chemical solvents react with acidic compounds such as CO₂ and H₂S.

Suitable physical solvents include tetramethylene sulphone (sulpholane) and derivatives, amides of aliphatic carboxylic acids, N-alkyl pyrrolidone, in particular N-methyl pyrrolidine, N-alkyl piperidones, in particular N-methyl piperidone, methanol, ethanol, ethylene glycol, polyethylene glycols, mono- or di(C₁-C₄)alkyl ethers of ethylene glycol or polyethylene glycols, suitably having a molecular weight from 50 to 800, and mixtures thereof. The preferred physical solvent is sulfolane. It is believed that CO₂ and/or H₂S are taken up in the physical solvent and thereby removed. Other treatments may include a further compression, when the purified gas is wanted at a higher pressure. Alternatively, the purified gas may be subjected to one or more further cooling and separation steps as described above. In this case the gas stream is subsequently subjected to a total number of combinations of subsequent cooling and separation steps. This number may suitably vary from 2 to 5 combinations.

In the event that the hydrocarbon gas is natural gas, the purified natural gas can be processed further in known manners, for example by catalytic or non-catalytic combustion to produce synthesis gas, to generate electricity, heat or power, or for the production of liquefied natural gas (LNG), or for residential use. It is an advantage of the present process enables purification of natural gas comprising substantial amounts of acidic contaminants, resulting in purified natural gas comprising low levels of contaminants, especially of sulphur contaminants. The production of LNG from such natural gas, which would be very difficult if not impossible by conventional processes, is made possible. Thus, the invention also provides LNG obtained from liquefying purified natural gas obtained by the process. The LNG thus-obtained typically has very low concentrations of contaminants other than natural gas.

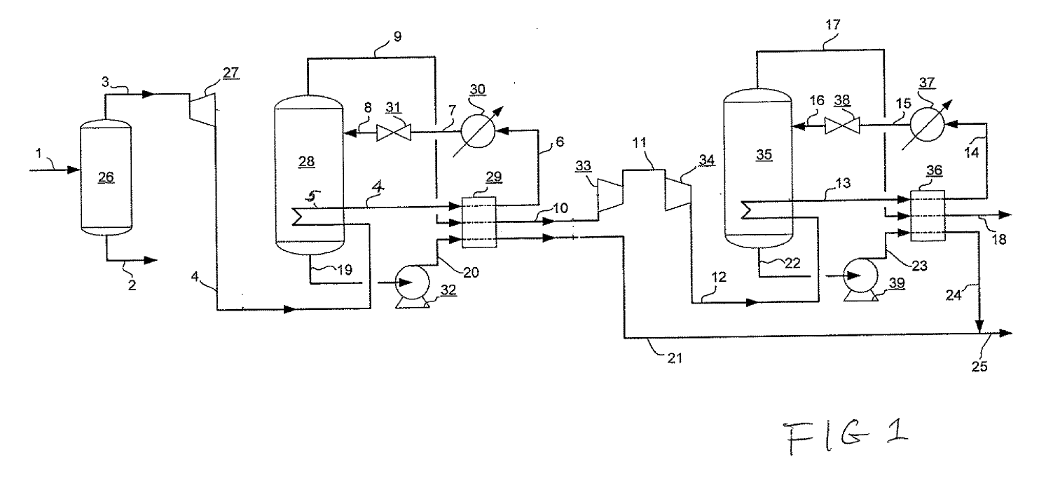

### FIG. 1:

The present invention will be further illustrated by means of the following figure.

In the description of FIG. 1 reference is made to a natural gas stream as an example of the gas stream that may be treated in the process according to the present invention. FIG. 1 shows a schematic flow scheme of an embodiment according to the invention.

A natural gas stream is introduced via a line 1 into a dehydrating unit 26. In the dehydration unit water is being removed from the natural gas stream, e.g., by means of molecular sieves. The water is eventually removed via a line 2. The dehydrated natural gas is passed via a line 3 to a turbo-expander 27 where it is cooled, and subsequently forwarded via a line 4. The line 4 comprises a bundle coil 5 that is located in a vessel 28. In the vessel 28 the bundle coil 5 acts as a heat exchanger for solid acidic contaminants that are collected in the bottom of vessel 28, thereby melting solid acidic contaminants.

The natural gas in line 4 is cooled further. Via a heat exchanger 29 the natural gas stream is passed via a line 6 to a further optional heat exchanger 30. Via a line 7 the further cooled natural gas stream is passed to a Joule-Thomson valve 31 where it is cooled to a first temperature at which acidic contaminants solidify so that a slurry of acidic contaminants and liquid hydrocarbons fall down in the vessel 28 and partly purified gas is withdrawn at the top via a line 9. The figure shows that short line 8 connects the Joule-Thomson valve with the vessel 28. This line is typically short so that blocking of the line by solids is prevented. It is also possible to do away with the line altogether and connect the Joule-Thomson valve directly to the wall of vessel 28.

The slurry in the bottom of vessel 28 is heated by the natural gas stream that flows through the bundle coil 5, thereby melting solid acidic contaminants. The bundle coil is just an example of a way to heat and melt the solid acidic contaminants. Other heating means are also possible. One may use an electric immersion heart, as suggested in WO-A 2007/030888. One may also add relatively warm natural gas liquids to the solid acidic contaminants, as suggested in WO-A 2004/070297. A preferred way is to heat at least part of the liquid that is withdrawn from the vessel 28 via line 19 and recycle thus heated contaminants, which may be liquid or vaporous, into the vessel 28. Combinations of any of these heating means are also possible.

The line 19 from the bottom of the vessel 28 leads the melted contaminants to an optional pump 32, and via a line 20 and heat exchanger 29 the contaminants are withdrawn through a line 21. In heat exchanger 29 the cold contaminants in line 20 and cold partly purified gas in line 9 are subjected to heat exchange with the natural gas stream in line 5. The streams are shown in co-current fashion. It is evident to the skilled person that the streams may also be arranged in a counter-current way, e.g., the relatively warm natural gas steam in counter-current with the two other streams. It will be appreciated that it is also feasible to use only one of the other streams or use a stream from another process, such as a stream from an LNG plant and/or an air separation plant.

From the heat exchanger 29 the partly purified gas is passed via a line 10 to a compressor 33. The compression energy for compressor 33 is suitably provided by the expander 27. The compressed gas may be passed to the second cooling stage from compressor 33. Optionally, when higher pressures are desired, the compressed gas may first be brought to a still higher pressure by means of a second compressor 34 fed with the partly purified gas provided by line 11. Via a line 12 the compressed partly purified gas is cooled in a bundle coil 13 in a second vessel 35. From the tube bundle coil 13 the gas is passed via a heat exchanger 36 and, optionally via a further heat exchanger 37, to a Joule-Thomson valve 38 where the gas is expanded and cooled to the second temperature. The cooled gas is then fed via a line 16 to the vessel 35, where solid acidic contaminants fall to the bottom together with any liquid contaminants and/or liquid hydrocarbons. Just like line 8, it is also feasible to shorten line 16 or delete line 16 to connect the valve to the wall of vessel 35. The bundle coil 13 heats up the solid acidic contaminants to melt them. Via a line 22 at the bottom of vessel 35 the liquid contaminants and any liquid hydrocarbons that may be present, are withdrawn, and by means of a pump 39 removed from the process via a line 23 and the heat exchanger 36. The contaminants may be combined with the contaminants in line 21 via a line 24 and together removed from the process for further treatment, storage or use in enhanced oil recovery.

The purified hydrocarbon gas is removed from the vessel 35 and also via a line 17 and the heat exchanger 36 recovered as product. Similar to heat exchanger 29 also heat exchanger 36 may be provided in a co-current or a counter-current fashion.

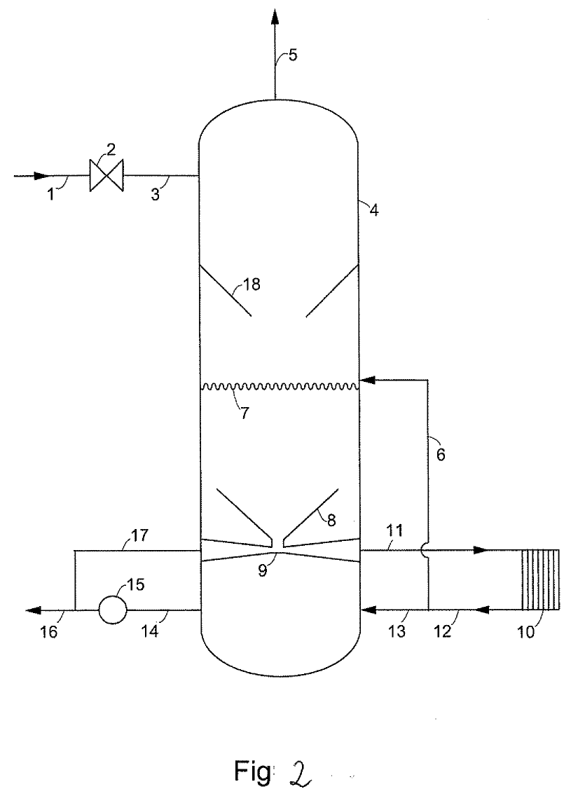

### FIG. 2

In one embodiment of vessels 28 and/or 35 in FIG. 1 is shown. Natural gas is passed via a conduit 1 through an expansion means 2, especially a Joule Thomson valve, whereby a stream is obtained of a slurry which comprises solid contaminant, liquid phase contaminant and a methane enriched gaseous phase. The stream of the slurry flows via a conduit 3 into cryogenic separation vessel 4. A methane enriched gaseous is removed from the separation vessel via a conduit 5. A stream of liquid phase contaminant is introduced into the separation device via a conduit 6 to dilute the slurry inside the separation device, establishing or maintaining a slurry level 7. The diluted slurry of contaminated is directed by means of a funnel 8 towards the top opening of an eductor 9. In the eductor 9 the diluted slurry is used as a suction fluid and via the eductor 9 it is passed into a heat exchanger 10 via a conduit 11. In the heat exchanger 10 solid contaminant present in the diluted slurry is melted into liquid phase contaminant. Part of the liquid phase contaminant so obtained is passed via a conduit 12 to the conduit 6, whereas the main part of liquid phase contaminant is introduced into the bottom part of the separation vessel 4 by means of a conduit 13. Liquid phase contaminant is subsequently withdrawn from the separation vessel 4 by means of a conduit 14 using a pump 15. Part of the withdrawn liquid phase contaminant is recovered as a product stream via a conduit 16 and part of said liquid phase contaminant is recycled via a conduit 17 to the eductor 9. A funnel 18 is present to guide the slurry stream into the direction of funnel 18.

1. Process for producing purified hydrocarbon gas from a gas stream comprising methane and acidic contaminants, which process comprises the steps of:
(a) cooling the gas stream in a first cooling stage to a first temperature to form a first mixture of solid and/or liquid acidic contaminants and a vapour containing gaseous hydrocarbons and a reduced amount of acidic contaminants;
(b) separating the solid and/or liquid acidic contaminants from the first mixture, yielding partly purified gas;
(c) cooling the partly purified gas in a second cooling step to a second temperature to form a second mixture comprising purified hydrocarbon gas and further solid and/or liquid acidic contaminants; and
(d) separating the further solid and/or liquid acidic contaminants from the second mixture, yielding the purified hydrocarbon gas.

2. Process as claimed in claim 1 in which the second temperature in the second cooling stage is lower than the first temperature in the first cooling stage.

3. Process as claimed in claim 2, in which the temperature difference between the first temperature and the second temperature is in the range of from 5 to 50° C.

4. Process as claimed in claim 1, in which the first temperature is from −40 to −80° C. and the second temperature is from −50 to −100° C.

5. Process as claimed in claim 1, in which the first cooling stage comprises one or more heat exchange steps.

6. Process as claimed in claim 5, wherein the gas stream is cooled to a temperature ranging from 1 to 40° C. above the freeze out temperature of the first acidic contaminant to freeze out, the freeze out temperature being the temperature at which solid contaminants are formed.

7. Process as claimed in claim 1, in which the first cooling stage comprises one or more expansion steps.

8. Process as claimed in claims 6, in which energy that is recovered at the expansion step or steps of the gas stream is used for the compression step or steps of the partly purified gas.

9. Process as claimed in claim 1, in which the partly purified gas is brought to a temperature ranging from 1 to 40° C. above the freeze out temperature of the first acidic contaminant to freeze out, the freeze out temperature being the temperature at which solid contaminants are formed.

10. Process as claimed in claim 9, in which the second cooling stage comprises one or more expansion steps.

11. Process as claimed in claim 10, in which partly purified gas is expanded from a pressure ranging from 30 to 100 bar, to a pressure of 5 to 30 bar.

12. Process as claimed in claim 1, wherein the purified gas is purified natural gas, the process further comprising the step of cooling the purified natural gas to obtain liquefied natural gas.
